(19) United States
(12) Patent Application Publication
(10) Pub. No.: US 2020/0323431 A1
(43) Pub. Date: Oct. 15, 2020

(54) IMAGING METHOD AND SYSTEM FOR INTRAOPERATIVE SURGICAL MARGIN ASSESSMENT

(71) Applicant: THE REGENTS OF THE UNIVERSITY OF CALIFORNIA, Oakland, CA (US)

(72) Inventors: Maie St. John, Los Angeles, CA (US); George Saddik, Newbury Park, CA (US); Zachary Taylor, Poway, CA (US); Warren Grundfest, Los Angeles, CA (US)

(73) Assignee: THE REGENTS OF THE UNIVERSITY OF CALIFORNIA, Oakland, CA (US)

(21) Appl. No.: 16/858,594

(22) Filed: Apr. 25, 2020

Related U.S. Application Data
(63) Continuation of application No. PCT/US2018/058806, filed on Nov. 1, 2018.
(60) Provisional application No. 62/580,383, filed on Nov. 1, 2017.

(51) Int. Cl. A61B 5/00

(52) U.S. Cl. CPC A61B 5/0071; A61B 5/4866; A61B 5/0082; A61B 2505/05; A61B 2562/0233; A61B 2562/04; A61B 2560/0223

(57) ABSTRACT

An imaging system and method is disclosed for intraoperative surgical margin assessment in between various tissues and cell groupings having differing physiologic processes. The system uses an array of LED's to pump a target anatomy with a short excitation pulse and measures the lifetime of fluorescence to generate contrast. A relative fluorescence lifetime map is generated corresponding to the measured lifetime to identify boundaries within varying cell groupings and tissues.

### CROSS-REFERENCE TO RELATED APPLICATIONS

This application claims priority to, and is a 35 U.S.C. § 111(a) continuation of, PCT international application number PCT/US2018/058806 filed on Nov. 1, 2018, incorporated herein by reference in its entirety, which claims priority to, and the benefit of, U.S. provisional patent application Ser. No. 62/580,383 filed on Nov. 1, 2017, incorporated herein by reference in its entirety. Priority is claimed to each of the foregoing applications.

The above-referenced PCT international application was published as PCT International Publication No. WO 2019/089998 A1 on May 9, 2019, which publication is incorporated herein by reference in its entirety.

### STATEMENT REGARDING FEDERALLY SPONSORED RESEARCHOR DEVELOPMENT

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to surgical imaging, and more particularly to interoperative surgical margin assessment.

### 2.Background Discussion

There is an unmet need for real-time methods to map tumor margins intraoperatively. Surgeons must accurately determine tumor margins intraoperatively to minimize over/under resection. This often results in: (a) under-resection (positive margins), which increases risk for disease recurrence; (b) over-resection (excessive negative margins), which can significantly reduce patient quality of life (e.g. reduced mobility, speech, etc.).

The clinician's fingertips (i.e. palpation) are the current gold standard for intraoperative margin assessment, which is subjective to each individual's touch. Other existing methods include: (a) time-consuming frozen sections that generally require a team of personnel; and (b) conventional ultrasound, CT, or MRI, which lack sensitivity and contrast.

For head and neck squamous cell carcinoma (HNSCC), only 67% of tumors are adequately excised, and local recurrence is 80% when margins are positive. This problem is seen in all cancers that undergo surgical removal.

Identification of other tissue types can also be problematic. For example, the variable location and indistinct external features of parathyroid glands can make their intraoperative identification challenging, especially when distinguishing them from adjacent fat or lymphatic tissue. Complications, such as hypo-parathyroidism and recurrent laryngeal nerve injury, are generally limited, but revision surgery and comprehensive explorations can increase operative morbidity. While preoperative imaging studies are available, real-time imaging methods that can efficiently localize parathyroid gland tissue in vivo, however, remain elusive.

### BRIEF SUMMARY

An aspect of the present disclosure is an imaging system and method for intraoperative surgical margin assessment in between various cell groupings having different physiologic processes, or differing tissues, for example but not limited to margins between any of pre-cancerous, pre-malignant, cancerous (e.g. oral and head and neck squamous cell carcinoma (OSCC)) and non-cancerous or benign (e.g. inflammatory) tissues or cell groupings. The imaging system and method use a technique herein referred to as time-resolved autofluorescence, which pumps a sample with a short excitation pulse and measures the lifetime of fluorescence (intensity of the emission as it decays from bright to dark) to generate contrast. A false color map, or like illustrative tool, may be generated corresponding to the measured lifetime. For tissue autofluorescence, naturally occurring fluorophores are used to create contrast (e.g. black light imaging). Information in the wavelength of emission.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1A shows a plot of raw values for normalized intensity over time.

FIG. 1B shows a plot of measured lifetimes.

FIG. 1C shows an exemplary lifetime map.

FIG. 1D shows normalized intensity across an array of pixels within the map of FIG. 1C.

FIG. 2 shows a schematic block diagram illustrating the various components of an exemplary DOCI system according to the present technology.

FIG. 3 shows a perspective view of the camera, lens and LED array of the system of FIG. 2.

FIG. 4 shows a cross-sectional view of a UV diode in accordance with the present description.

FIG. 5 shows a flow diagram of an algorithmic method for imaging a sample using the system of the present description.

FIG. 6 shows an embodiment of the LED array and corresponding dispersion of illumination via non-sequential ray tracing.

FIG. 7 shows an exemplary plot of target irradiation from an exemplary LED array in accordance with the system of the present description.

FIG. 8A is a plot of a simulated impulse response from an illumination pulse.

FIG. 8B is a plot of simulated fluorophore emissions.

FIG. 8C is a plot of simulated detected emissions with noise and an offset being introduced.

FIG. 8D is a simulation plot of the ratio of the calibration and decay image (calibrated by an offset due to dark current, A) and its value as a function of decay image gate width.

FIG. 9A shows an exemplary output fluorescence corresponding to the scalp tissue sample image of FIG. 9B.

FIG. 10A shows an exemplary output fluorescence corresponding to the tongue tissue sample image of FIG. 10B.

FIG. 11 shows a plot of computed relative lifetime as a function of wavelength for tumor, muscle, fat and collagen, demonstrating stark differences between each tissue type.

FIG. 12 is a plot illustrating statistical significance at various wavelengths for muscle, collagen and fat.

FIG. 13 is an in vivo image of patient mouth tissue.

FIG. 14 is an ex vivo H&E image of a portion of the region in FIG. 13.

FIG. 15A is a close-up, reconstituted RGB image of the tongue tissue of FIG. 13.

FIG. 15B through FIG. 15E show in vivo DOCI images at 407 nm, 434 nm, 465nm and 494 nm, respectively, of the field of view of the reconstituted image of FIG. 15A.

FIG. 16A shows a close-up portion of the reconstituted RGB image of FIG. 15A.

FIG. 16B through FIG. 16E show ex vivo images at 407 nm, 434 nm, 465 nm and 494 nm, respectively, having the same field of view of the image of 16A.

FIG. 17A shows a visible image of parathyroid tissue.

FIG. 17B shows a DOCI image of the tissue of FIG. 17A.

FIG. 17C shows a histology image of the tissue of FIG. 17A.

FIG. 18A is an image a subject's mouth having a lip with pre-cancerous cell physiology.

FIG. 18B is an image of a second subject's mouth having an inflamed lip (benign cell physiology).

FIG. 18C is the image of FIG. 18A overlaid with a DOCI image of the subject's lip.

FIG. 18D is the image of FIG. 18B overlaid with a DOCI image of the subject's lip.

### DETAILED DESCRIPTION

The systems and methods of the present description implements naturally occurring differences in fluorophore lifetime between cell groupings having different physiological processes are used to generate contrast, and unique algorithms are applied to relax technical requirements. For tissue autofluorescence, naturally occurring fluorophores are used to create contrast (e.g. black light imaging). In one embodiment, the target is illuminated with a short pulse of light and the intensity of the emission as it decays from bright to dark is measured. How long an area “glows” is determinant of what type of tissue was illuminated. For example, cancerous tissues are generally associated with fast decay, and non-cancerous tissues are associated with slow decay.

The systems and methods disclosed herein are configured for margin detection between cell groupings having different physiologic processes, or differing tissues, for example but not limited to margins between any of pre-cancerous, pre-malignant, cancerous (e.g. oral and head and neck squamous cell carcinoma (OSCC)) and non-cancerous or benign (e.g. inflammatory) tissues or cell groupings.

A. Systems and Methods

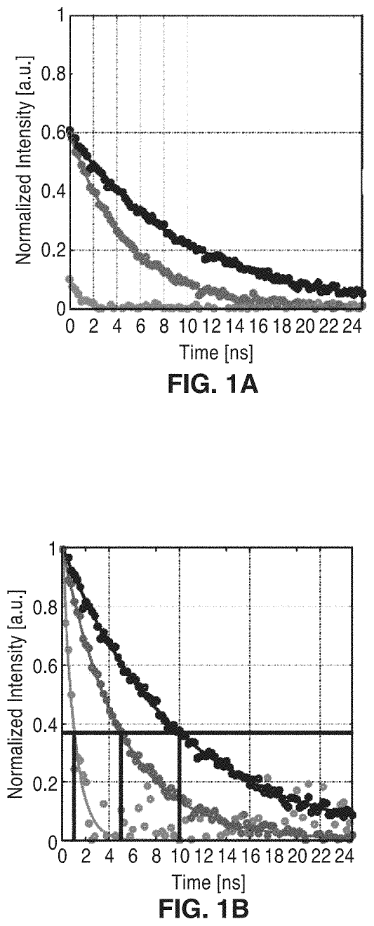

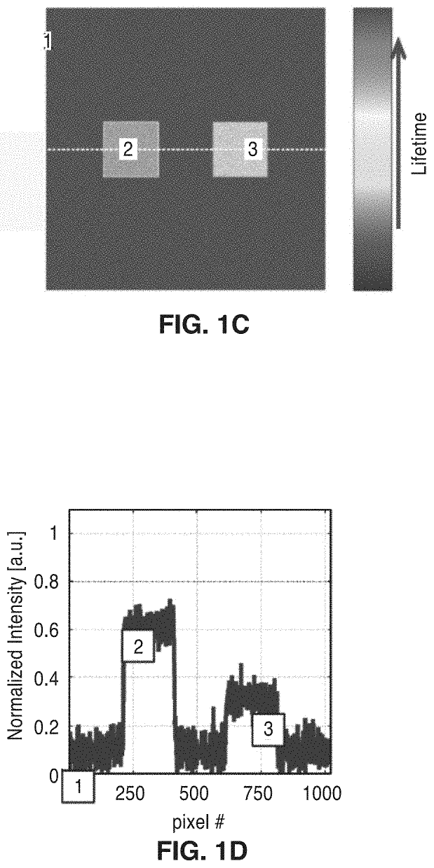

FIG. 1A through FIG. 1D illustrate an exemplary process for performing time-resolved autofluorescence in according with the present technology. To generate lifetime decay curves fluorescence is measured as a function of time. Fluorescence typically decays over a period of picoseconds to nanoseconds after an excitation pulse. The rate of decay (i.e. “lifetime”) of fluorescence at each point in the image is plotted as a distribution of fluorescence ‘lifetime’ values. In the presence of fluorophores, the slope of the decay curve is less steep due to the existence of a finite excited state. Thus, fluorophores with longer lifetimes are characterized by larger slopes. Based on the specific lifetime of the fluorophore, fluorescence between different tissues (e.g. normal and cancerous tissue) can be distinguished. FIG. 1A shows an exemplary plot of raw intensity values are first obtained, as shown in. FIG. 1B shows an exemplary plot of measured lifetimes. A lifetime map is then generated as shown in FIG. 1C. FIG. 1D shows normalized intensity across an array of pixels within the map of FIG. 1C. As compared to standard fluorescence, lifetime fluorescence has improved robustness to clutter, maximum contrast generation, and is ideal for in vivo imaging.

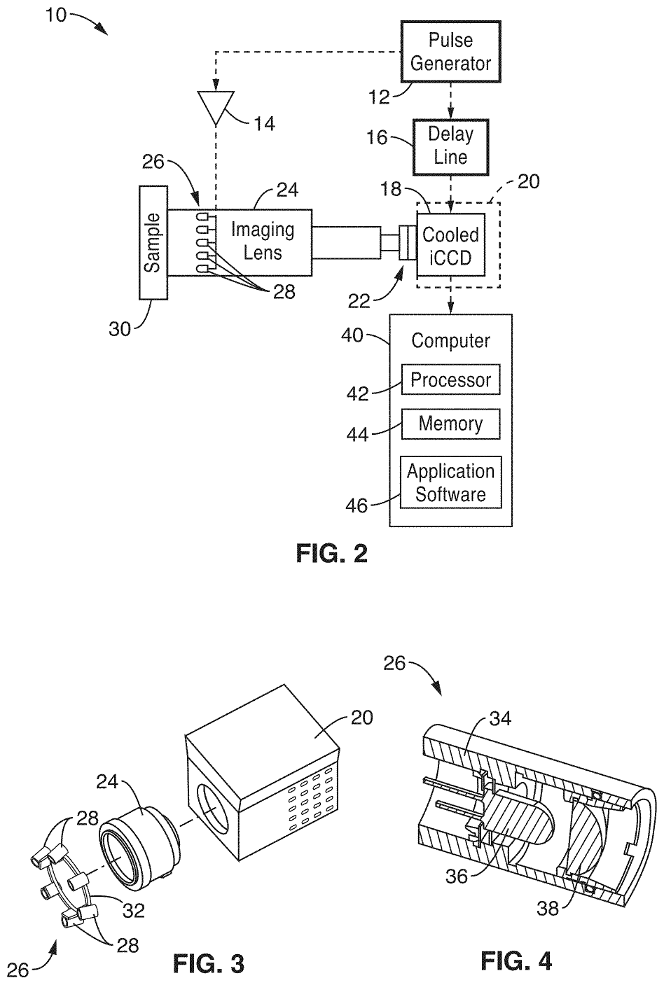

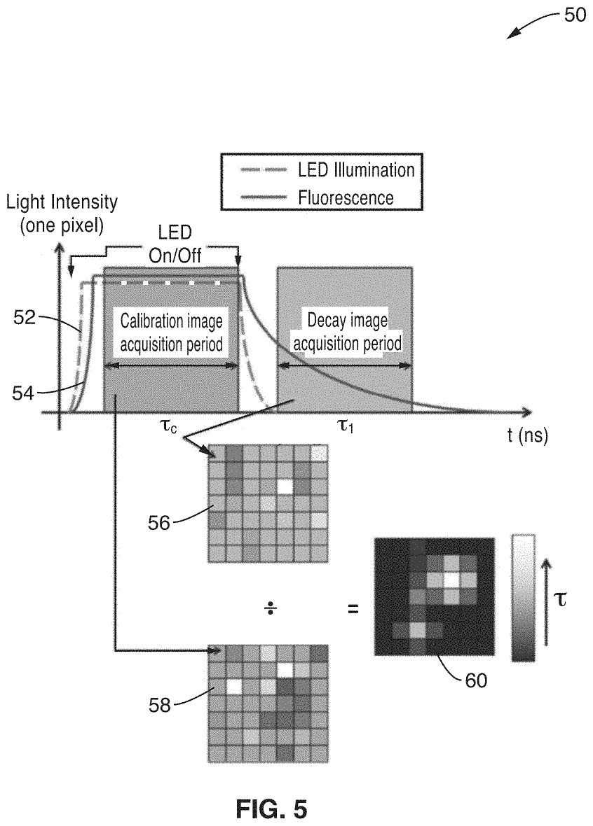

FIG. 2 shows a schematic block diagram illustrating the various components of an exemplary dynamic optical contrast imaging (DOCI) system 10 according to the present technology. In a preferred embodiment, the DOCI system 10 comprises an imaging lens 24, and an array 26 of UV diodes (LED's) 28 disposed at the front of the lens 24. The array 26 of UV diodes 28 is configured to illuminate sample 30 via a signal generated by pulse generator 12 and diode driver 14. Pulse generator 12 is also coupled to gated camera 20 via a delay line 16. Camera 20 preferably comprises a cooled iCCD 18, and UV laser line filters 22 disposed between the iCCD 18 and lens 24 (filters 22 may be disposed anywhere within the optical path). The LED array 26 and camera 20 output are coupled to a computer 40 (or like computing device) comprising a processor 42, application software 46, and memory 44 storing application software 46 for execution on processor 42. Application software 46 comprises instructions for operating the components of the system (e.g. pulse generator 12, LED array 26, diode driver 14, etc.) and processing the data acquired from iCCD 18 (e.g. instructions for performing method 50 detailed below and illustrated in FIG. 5).

Filters 22 may comprise a filter wheel configured to restrict light received by iCCD 18, so that only a certain wavelength or range of wavelengths is imaged at a given time. For example, a first image may be obtained with only red light, with second and third images being restricted to only blue and green light. These images may be displayed simultaneously in different panels for display, or combined, for example, to generate a reconstituted RGB image that may be used as a fiduciary image in conjunction with visualization (e.g. side-by-side display) of one or more generated DOCI images at various wavelengths (see FIG. 15A through FIG. 15C). In one embodiment, filters 22 comprises a filter wheel comprising 10 filters to restrict light centered at the following emission bands: 407 nm, 434 nm, 465 nm, 494nm, 520 nm, 542 nm, 572 nm, 605 nm, 632 nm, and 676 nm. It is appreciated that the above bands are for illustrative purposes only, and other variations are contemplated.

In one embodiment, the UV diode array 26 illuminates at a wavelength of 375 nm (this may be varied based on target tissue/device specifications). The light-emitting diode illumination circuit (diode driver 14) operates at a center wavelength of 370 nm, an average optical power of approximately 4.5 μW, and a pulse width of 30 ns. The low average power and the long wavelength ensures that proteins, DNA, and other molecules are not adversely affected by imaging.

FIG. 3 shows an exploded perspective view of the camera 20, lens 24 and LED array 26. In a preferred embodiment, the LED array 26 is aligned with the front of lens 24 such that the individual LEDs 28 are aligned circumferentially around the lens 24. Frame 32 holds the individual LEDs 28 in proper alignment and allows for coupling of the array 26 to the lens 24.

FIG. 4 shows a cross-sectional view of a UV diode 28 in accordance with the present description. Each UV diode 28 comprises a housing 30 configured to house a UV LED 36 and spherical lens 38 configure to shape the transmitted light for focused dispersion across the entire field of view, or significant portion thereof.

FIG. 5 shows a flow diagram of an algorithmic method 50 for imaging a sample 30 using the system 10 of the present description. Method 50 applies a unique image frame normalization scheme to produce pixel values that are proportional to the aggregate fluorophore of the probed tissue, without the requirement of fitting complex mathematical models to acquired data. This relaxes the requirements on the temporal profile of the illumination pulse and enables the replacement of picosecond pulsed lasers (that are generally necessary for FLIM) with nanosecond pulsed light emitting diodes (LEDs). Illumination is performed via UV a light source 26 (e.g. at 375 nm) in long pulse durations (˜30 ns) with short (nanosecond order) rise and fall times to produce contrast between fluorophores of difference decay rates. With this scheme, the scalable mapping of fluorophore lifetimes over macroscopic (not microscopic) fields of view (FOV) is possible within a relatively short time frame (˜10 seconds per emission band), with all pixels acquired simultaneously. These improvements thus provide a significant step towards intraoperative clinical use.

As seen in FIG. 5, two gates are acquired, one calibration acquisition period Tc and one decay image acquisition period τ₁. The fluorescence lifetimes of most tissue constituents of interest in head and neck imaging fall within the range of 1 ns to 10 ns. Therefore, an emission image, acquired at >10 ns following initial illumination, can be considered as an accurate representation of steady state tissue autofluorescence. To calibrate the fluorescence emissions acquired, an image is captured mid-UV pulse duration at a calibration acquisition period τ_{c} to generate image 58, hereinafter referred to as “FOV calibration image.” Subsequently, at the beginning of illumination pulse decay (decay image acquisition period τ₁), a second image 56 is captured, hereinafter referred to as “FOV decay image.”

In FIG. 5, dashed line 52 represents the LED intensity during on/off stages, and solid line 54 shows the acquired fluorescence intensity (for each individual pixel). The FOV decay image 56 is then is then normalized (pixel wise by the calibration image) by dividing the FOV decay image 56 by the FOV calibration image 58 to generate the FOV relative lifetime map 60. In a preferred embodiment, images may be acquired at numerous wavelengths (e.g. via filter 22 may comprise a filter wheel or like device that allows for selection from among a number of different wavelength ranges allowing certain wavelengths to be received by camera 20).

In a preferred embodiment, the FOV decay image 56 and the resulting pixel values are proportional to the aggregate fluorophore decay time of the illuminated area. These pixel values represent relative tissue lifetimes and are referred to as DOCI pixel values. DOCI relies on the fact that the longer lifetime fluorophores generate more signal than shorter lifetime fluorophores when referenced to their steady state fluorescence. It is also appreciated that additional images (e.g. background image or the like) may be obtained to further process and generate the relative lifetime map 60.

The relative lifetime map 60 may be displayed as a false color map, or as any visual representation of quantitative relative lifetime pixel values in lines, shapes, colors, or auditory cues to the operator.

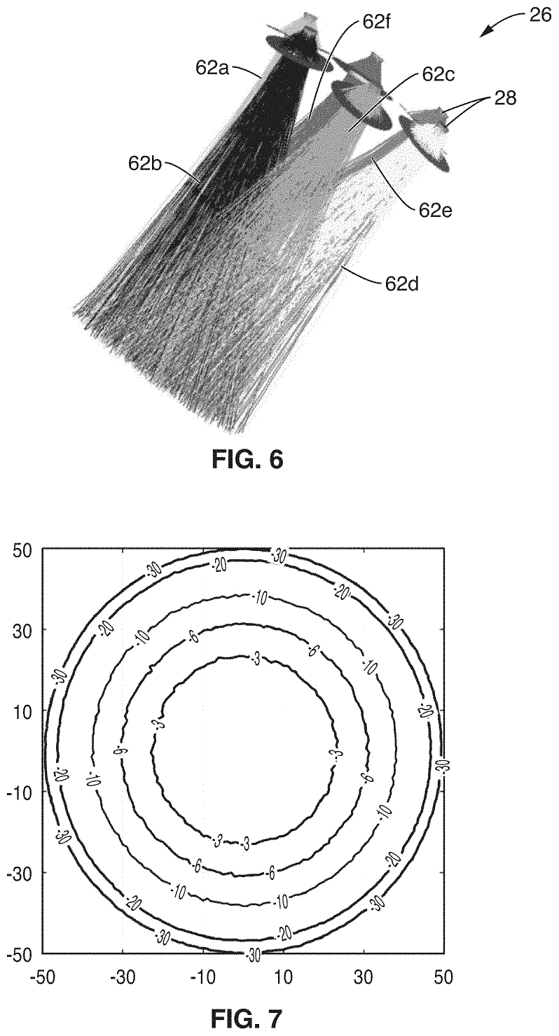

FIG. 6 shows an embodiment of the present LED array 26 illustrating non-sequential ray tracing via illumination beams 62a through 62f from individual LEDs 28 to focus and multiply the excitation light from each LED across the FOV. In a preferred embodiment, the pattern of the non-sequential ray tracing is shaped and Illumination distribution and intensity adjustments that are varied according to selection of LED bulb 36 and lens 38 (FIG. 4). FIG. 7 shows an exemplary plot of target irradiation resulting from an exemplary LED array 26 and resulting ray tracing illumination pattern.

B. DOCI: Principles of Operation

For purposes of this analysis, an illumination pulse is modeled as an ideal rectangular pulse convolved with the impulse response of a single pole low pass filter to model the band limit of the illumination pulse FIG .8A. A single time constant exponential impulse response is described in Eq. 1:

hₖ(t)=u(t)e^{-t/τ}^{ₖ}, Eq. 1

where τₖ=τ_{d} (illumination time constant), τ₁ (fluorophore 1 time constant), or τ₂ (fluorophore 2 time constant).

This illumination profile is described in Eq. 2:

x_{d}(t)=(Π_{T}_{ₒ}h_{d})(t), Eq. 2

where T₀ is the pulse width.

Fluorophore specific lifetimes can therefore be modeled with Eq. 1 The fluorescence emission of the UV pumped fluorophores is written as the convolution of the diode illumination and fluorescence decay times according to Eq. 3:

y_{1,2}(t)=(h_{1,2}*x_{d})(t) Eq. 3

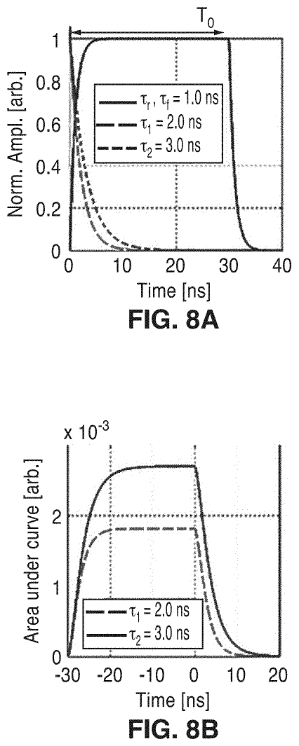

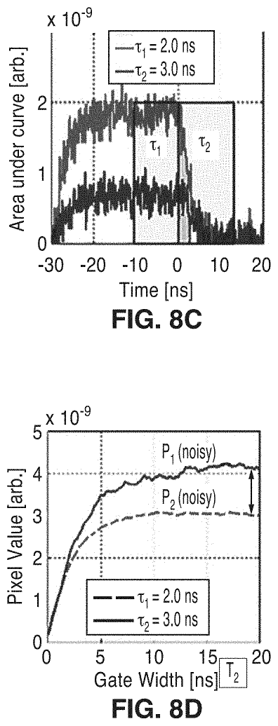

A graphical representation of these convolution integrals is shown in FIG. 8B where the individual traces are the fluorescence emissions of fluorophores 1 and 2, respectively.

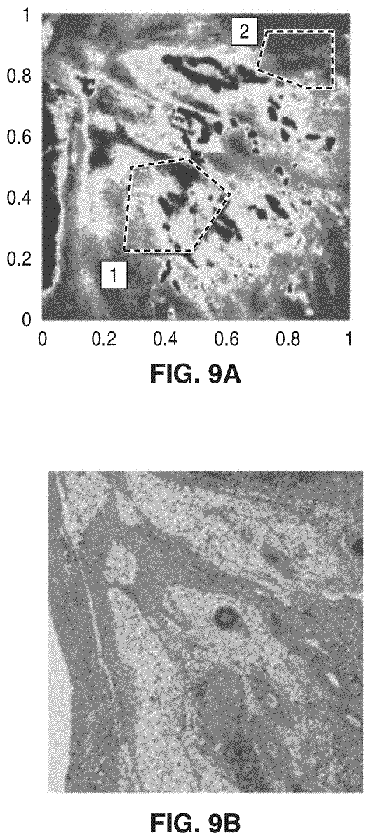

Next, band limited white Gaussian noise and an offset (due to dark current) is introduced, the output of which is shown in FIG. 8C. As an additional image confounder, pixels harboring the fluorophores of interest are subjected to 1) effects of illumination and 2) blocking obscurants chosen arbitrarily as a 90% drop in detected fluorescence emission in fluorophore 1 and a 97.5% drop in fluorophore 2. These results are integrated into the output shown in the image of FIG. 9C. This combination of fluence absorption, uncorrelated white measurement noise, and dark current reduces the peak SNR of the received intensity of fluorophore 2 to 6 dB. (The time axes of FIG. 8B to FIG. 8D are defined such that the illumination/emission decay occurs at t=0).

A calibration measurement is acquired just before the illumination pulse begins to decay with a gate width of T₁. This process, illustrated in FIG. 8C, is described in Eq. 4:

$${C}_{1 , 2} = \frac{1}{{T}_{1}} {\int }_{- {T}_{1}}^{0} {A}_{1 , 2} {y}_{1 , 2} ( t ) + n ( t ) d t \qquad Eq . 4$$

The decay measurement undergoes similar acquisition methodology described by Eq. 5 (also shown in FIG. 8C) using gate width T₂:

D_{1,2}=∫₀^{T}^{₂}A_{1,2}y_{1,2}(t)+n(t)dt Eq. 5

The resultant DOCI pixel value is calculated according to Eq. 6:

$${P}_{1 , 2} = \frac{{D}_{1 , 2} - Δ}{{C}_{1 , 2} - Δ} , \qquad Eq . 6$$

and is the ratio of the calibration image and decay image (calibrated by an offset due to dark current, A) and its value as a function of decay image gate width, and is illustrated in FIG. 8D, where increasing the gate length increases the difference signal computed between the two fluorophores and both signals converge (ideally) to the sum of the illumination and fluorophore decay times.

One strength of the DOCI system and methods is that it converts fluorophore lifetime into contrast by computing the area under the decay time curve normalized to the steady state fluorescence. In the limit of stationary noise, this process is robust to variations in obscurants and can produce significant contrast under low SNR.

This approach has many key advantages that make it ideal for clinical imaging. First, as discussed above, the computational technique is simple; lifetimes are not calculated, therefore curve fitting is not required. Second, relaxed lifetime calculations allow for longer pulse duration intervals and fall times (>1 ns); thus, cheap LEDs driven by electronic pulses may be used instead of expensive lasers. Third, the difference in signal between the emission decay of two fluorophores is positively correlated with gate time. In other words, the longer the gate is open during the decay image, the larger the difference signal. In addition, the signal to noise ratio (SNR) significantly increases due to increased signal and decreased measurement noise arising from the integrative properties of the detector. This is in stark contrast to FLIM where gates need to be short to accurately sample the decay time. Rather, for the DOCI process, contrast is enhanced when the gate width is increased as it increases the overall number of collected photons while reducing noise variance. The simplicity and intrinsic sensitivity of the technology enables rapid imaging of large FOVs practical for clinical imaging.

C. Experimental Results

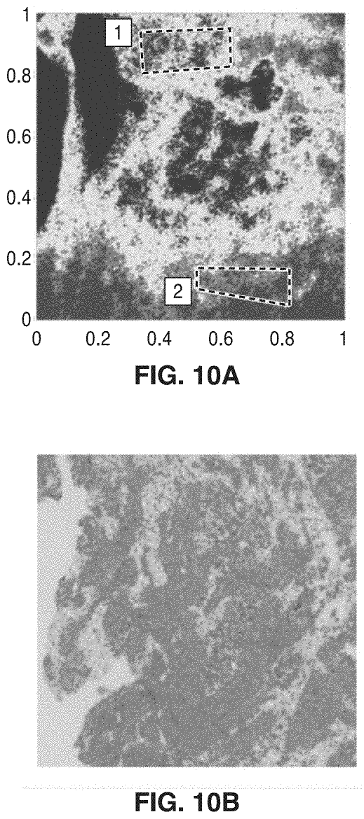

A number of ex vivo trials were performed via fresh tissue trials (over 84 patients and 190 distinct images to demonstrate the efficacy of the DOCI system and methods detailed above. FIG. 9A shows an exemplary output fluorescence corresponding to the scalp tissue sample image of FIG. 9B. Dashed region 1 corresponds to tumor tissue, whereas dashed region 2 corresponds to muscle tissue. FIG. 10A shows an exemplary output fluorescence corresponding to the tongue tissue sample image of FIG. 10B. Dashed region 1 corresponds to tumor tissue, whereas dashed region 2 corresponds to muscle tissue.

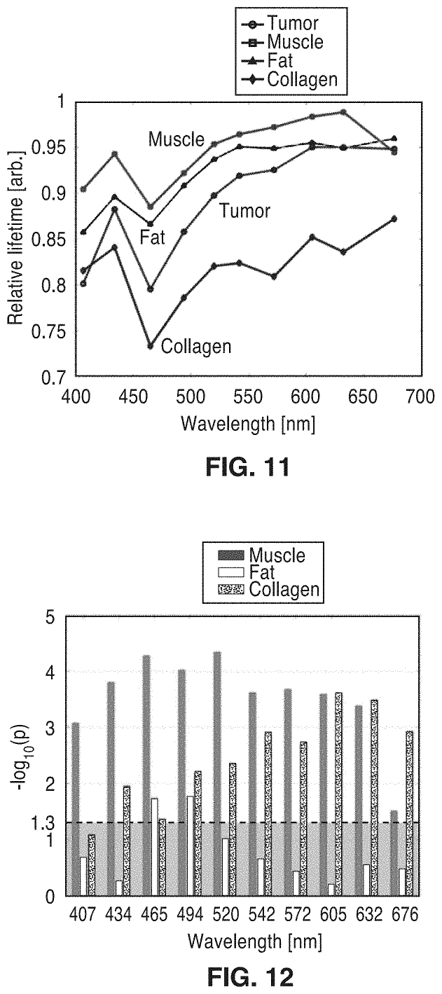

FIG. 11 shows a plot of computed relative lifetime as a function of wavelength for tumor, muscle, fat and collagen. The results demonstrate that DOCI lifetime mapping produced statistically significant differences in contrast between all four tissue types under investigation (tumor, fat, muscle, and collagen) across most emission wavelengths. A decrease in fluorescence lifetime was observed in malignant tissue and is consistent with short lifetimes reported for biochemical markers of tumors.

FIG. 12 is a plot illustrating statistical significance at various wavelengths for muscle, collagen and fat. Statistical significance (P<0.05) between muscle and tumor was established for 10 of 10 emission wavelengths, between collagen and tumor for 8 of 10 emission wavelengths, and between fat and tumor for 2 of 10 wavelengths. This study demonstrates the feasibility of DOCI to accurately distinguish OSCC and surrounding normal tissue and its potential to maximize the efficacy of surgical resection.

To evaluate the diagnostic utility of DOCI in the intraoperative detection of OSCC, an in vivo study of 15 consecutive patients undergoing surgical resection for OSCC was performed. Biopsy-proven squamous cell carcinoma neoplasms were obtained from the following head and neck sites and sub-sites: auricle, parotid, scalp, oral cavity, oropharynx, hypopharynx, and neck. All specimens were imaged with the DOCI system 10 (FIG. 2) prior to resection. Following tumor ablation, specimens were immediately sectioned into multiple fresh samples containing tumor and contiguous normal tissue of suspect lesions and submitted for histological assessment. Areas of neoplasm were then confirmed by a pathologist, blinded to the DOCI image results, and relative lifetime values were computed independent of pathologic diagnosis.

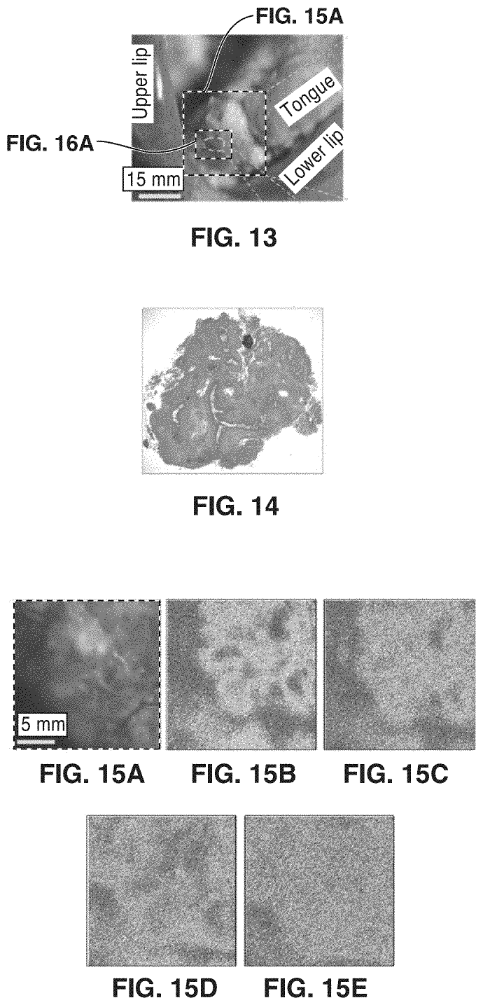

DOCI and visible images of a tongue OSCC are displayed in FIG. 13 through FIG. 16E. In a preferred embodiment of the DOCI systems and methods of the present disclosure, a DOCI image color map transforms blue to the global minimum relative decay lifetime and red to the maximum relative decay lifetime. A reduced DOCI pixel value indicates a more rapid decay of fluorescence signal, indicating an overall shorter lifetime. While the DOCI images depicted variably in FIG. 15B through FIG. 18E are provided in grey scale, it is appreciated that the lighter aspects in the DOCI images corresponds to the minimum relative decay lifetime (blue), and darker aspects in the DOCI images corresponds to the maximum relative decay lifetime (red).

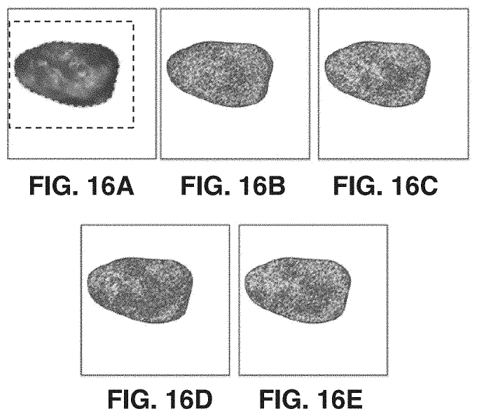

FIG. 13 is an in vivo image of a patient mouth tissue, while FIG. 14 shows an ex vivo H&E image of a portion of the region in FIG. 13. FIG. 15A shows a close-up, reconstituted RGB image of the tongue tissue of FIG. 13. FIG. 15B through FIG. 15E show in vivo DOCI images at 407 nm, 434 nm, 465 nm and 494 nm, respectively, of the field of view of the reconstituted image of FIG. 15A. FIG. 16A shows a close-up portion of the reconstituted RGB image of FIG. 15A. FIG. 16B through FIG. 16E show ex vivo DOCI images of the tongue image portion of FIG. 16A at 407 nm, 434 nm, 465 nm and 494 nm, respectively, having the same field of view of the image of 16A.

In a preferred embodiment application software 46 (FIG. 2) may be configured to output a reconstituted RGB image (FIG. 15A) simultaneous with (e.g. side-by-side as individual panels for display) one or more DOCI images at one or more wavelengths (e.g. FIG. 15B through FIG. 15E). To obtain the reconstitute RGB image, a first (non-DOCI) image may be obtained with only red light (e.g. via selection of appropriate filter on filter wheel 22 (FIG. 2)), with second and third images being restricted to only blue and green light (the iCCD 18 generally comprises one bin for data acquisition (as opposed to a multiple-bin RGB detector)). These images may be combined or fused to generate a reconstituted RGB image (FIG. 15 B) that may be used as a real-time fiduciary image in conjunction with visualization (e.g. side-by-side display) of one or more generated DOCI images at various wavelengths (see FIG. 15A through FIG. 15C) that are obtained via the same detector 18.

As shown in FIG. 15B through FIG. 15E, the in vivo DOCI images demonstrated a stark contrast between OSCC tissue and the surrounding normal tissue. Areas of OSCC were characterized by reduced relative lifetime compared to the lifetime of surrounding normal tissue.

Comparable relative lifetime measurements were observed in the post-resection ex vivo images of excited tissue (FIG. 16B and FIG. 16C). Strong positive correlations between ex-vivo OSCC and in-vivo OSCC over the entire range of emission wavelengths suggest that DOCI and its associated image analysis methods are directly translatable to in vivo clinical use. As DOCI images are acquired from the epithelial surface in vivo, DOCI images of ex-vivo specimens acquired along the same imaging plane are clinically relevant and revealed details on tissue structure/type that are not evident on gross examination. Ex-vivo tissue and corresponding histology were sectioned parallel to the imaged plane and epithelial surface to capture both cancerous and adjacent stroma. Differences in relative lifetimes were reported between areas of tumor, fat, muscle, and collagen that were assessed with DOCI.

The DOCI system and methods were also investigated for real-time, in vivo use for parathyroid localization. Ex vivo DOCI data in parathyroid tissue demonstrates potential for making this technology a reliable in vivo technique to produce a “relative decay map” of tissues, depicting an intra-operative color atlas that correspond to parathyroid gland location. A prospective series of patients (n=81) with primary hyperparathyroidism were examined. Parathyroid lesions and surrounding tissues were collected; fluorescence decay images were acquired via DOCI; and individual ex vivo specimens (n=127 samples) were processed for histologic assessment. Hand-delineated regions of interest (ROIs) were determined by histopathologic analysis and superimposed onto the corresponding high-definition visible images. Visible images were then manually eroded and registered to companion DOCI images. Finally, ROIs were averaged from fat (n=43), parathyroid (n=85), thymus (n=30), and thyroid tissue (n=45).

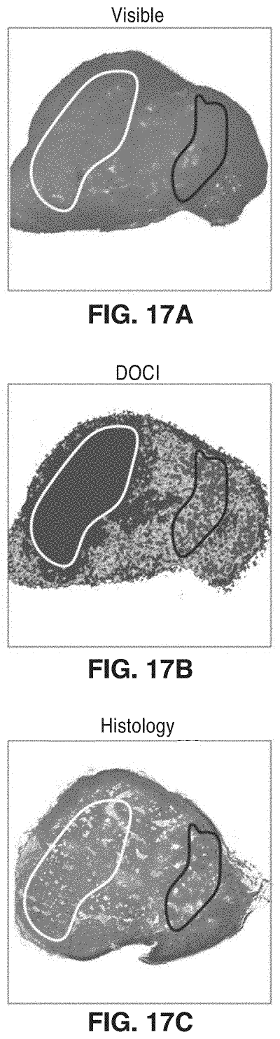

FIG. 17A through FIG. 17C show one example of parathyroid tissue sampled from the ex vivo trial, demonstrating DOCI contrast across the entire FOV. As observed in the images, parathyroid tissue displayed decreased relative lifetimes when compared to fat across all the filters utilized. Contrast between tissue types and cell groupings having different physiologic processes (e.g. parathyroid vs thyroid cell groupings) was evident across all the emission wavelengths. The basis of tissue contrast in the DOCI parathyroid images is likely due to the presence of hormone-specific proteins, amino acids, and extra-cellular calcium-sensing receptors within the densely populated parathyroid gland chief cells. This ex vivo DOCI data demonstrates efficacy for applying the technology of the present description a reliable in vivo technique to produce a “relative decay map” of parathyroid tissues, depicting an intra-operative color atlas that corresponds to parathyroid gland location.

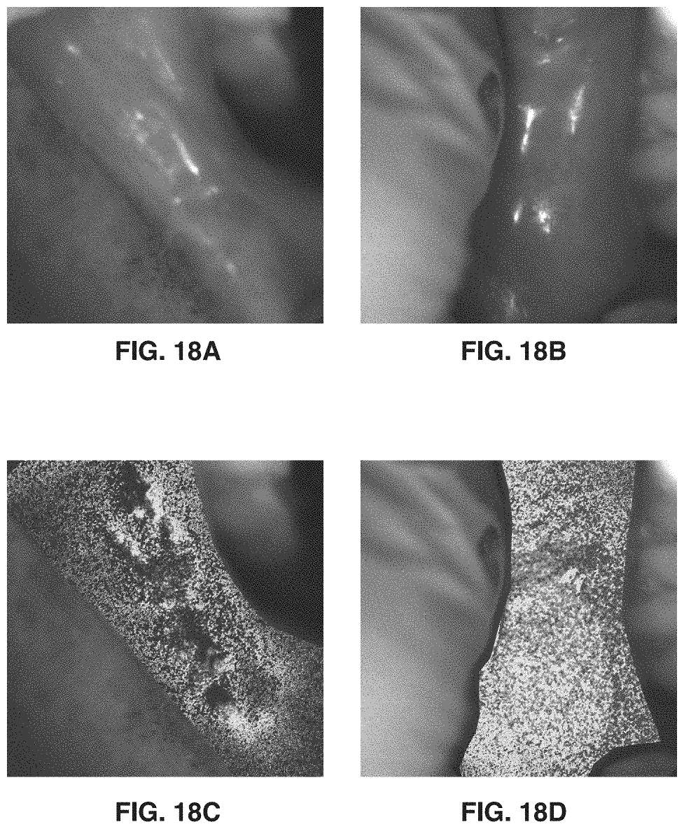

Referring to FIG. 18A through FIG. 18D, studies were also performed for in vivo detection of pre-cancerous or pre-malignant tissues or cell groupings via imaging of oral carcinoma. The DOC system and methods were able to provide differentiation of acitinic cheilitis/erythroplakia (pre-cancerous lesion) in one patient and inflammation in the lip (e.g. from sunburn or like benign condition) in another. Images were obtained depicting a visible image (FIG. 18A) with a DOCI overlay of the pre-cancerous lesion on the lip (FIG. 18C), compared with a visible image (18B) with DOCI overlay of inflammation of the lip (FIG. 18D).

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general-purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

From the description herein, it will be appreciated that the present disclosure encompasses multiple embodiments which include, but are not limited to, the following:

1. An apparatus for boundary detection within a target anatomy, comprising: (a) a processor; and (b) a non-transitory memory storing instructions executable by the processor; (c) wherein said instructions, when executed by the processor, perform steps comprising: (i) illuminating the target anatomy with an excitation pulse of light to excite fluorophores corresponding to the first tissue and second tissue; (ii) acquiring a calibration image of the target anatomy during the excitation pulse, the calibration image comprising fluorescence values from emissions of the excited fluorophores; (iii) acquiring a decay image of the target anatomy subsequent to the excitation pulse, the decay image comprising decayed fluorescence values as the emissions decay from bright to dark; (iv) dividing the decay image by the calibration image to generate a relative lifetime map of the target anatomy; and (v) using values in the relative lifetime map, identifying a boundary between a first group of cells having a first physiologic process and a second group of cells having a second physiologic process.

2. The system, apparatus or method of any preceding or subsequent embodiment, wherein identifying a boundary comprises identifying a transition between cells of different aggregate type or metabolic profile.

3. The system, apparatus or method of any preceding or subsequent embodiment, wherein identifying a boundary comprises identifying a transition between pre-cancerous cells and benign cells.

4. The system, apparatus or method of any preceding or subsequent embodiment, wherein identifying a boundary comprises identifying a transition between cancerous cells and non-cancerous cells.

5. The system, apparatus or method of any preceding or subsequent embodiment: wherein the calibration image and decay image comprise an array of pixels across a field of view (FOV) of the target anatomy; and wherein the pixels in the array of pixels comprise fluorescence lifetime values that are acquired simultaneously across the FOV for both the calibration image and the decay image.

6. The system, apparatus or method of any preceding or subsequent embodiment, wherein said instructions, when executed by the processor, perform steps comprising: generating a reconstituted RGB image of the target anatomy; and displaying the reconstituted image simultaneously with the relative lifetime map of the target anatomy.

7. The system, apparatus or method of any preceding or subsequent embodiment, wherein the reconstituted RGB image is generated by acquiring separate images of the target anatomy by limiting acquisition of each image to only red, blue and green wavelengths within successive image captures, and then combining separate red, blue and green image captures to form the reconstituted RGB image.

8. The system, apparatus or method of any preceding or subsequent embodiment, wherein the relative lifetime map comprises a false color map of normalized fluorescence lifetime intensity across the array of pixels within the relative lifetime map.

9. The system, apparatus or method of any preceding or subsequent embodiment, wherein the relative lifetime map comprises pixel values that are proportional to an aggregate fluorophore decay time of the FOV.

10. The system, apparatus or method of any preceding or subsequent embodiment, wherein the FOV comprises a macroscopic FOV of the target anatomy.

11. The system, apparatus or method of any preceding or subsequent embodiment, wherein the excitation pulse comprises a pulse duration of approximately 30 ns.

12. The apparatus of claim 1, further comprising: (d) an imaging lens; (e) an array of LEDs disposed at the front of the lens; (f) wherein the array of LEDs is configured to illuminate target anatomy with the excitation pulse of light for a specified duration, wherein the array of LEDs focuses and multiplies illumination of the target anatomy across a FOV of the imaging lens; and (g) a detector coupled to the imaging lens, the detector configured to acquire intensity data of the fluorescence emissions.

13. The system, apparatus or method of any preceding or subsequent embodiment, wherein each of the LEDs in the array of LEDs comprises an aspherical lens to focus the excitation pulse of light across the FOV.

14. The apparatus of claim 12, further comprising: (h) a diode driver coupled to the LED array; and (i) a pulse generator coupled to the diode driver and processor; (j) wherein the diode driver, pulse generator and LED array are coupled such that each of the array of LED's is configured to illuminate the FOV via non-sequential ray tracing.

15. A system for boundary detection within a target anatomy, the system comprising: (a) an imaging lens; (b) an array of LEDs disposed at or near the imaging lens; (c) a detector coupled to the imaging lens, the detector configured to acquire intensity data of fluorescence emissions from the target anatomy; (d) a processor coupled to the detector; and (e) a non-transitory memory storing instructions executable by the processor; (f) wherein said instructions, when executed by the processor, perform steps comprising: (i) operating the array of LEDs to illuminate the target anatomy with an excitation pulse of light to excite fluorophores corresponding to the first tissue and second tissue; (ii) acquiring a calibration image of the target anatomy during the excitation pulse, the calibration image comprising fluorescence values from emissions of the excited fluorophores; (iii) acquiring a decay image of the target anatomy subsequent to the excitation pulse, the decay image comprising decayed fluorescence values as the emissions decay from bright to dark; (iv) dividing the decay image by the calibration image to generate a relative lifetime map of the target anatomy; and (v) using values in the relative lifetime map, identifying a boundary between a first group of cells having a first physiologic process and a second group of cells having a second physiologic process.

16. The system, apparatus or method of any preceding or subsequent embodiment wherein identifying a boundary comprises identifying a transition between cells of different aggregate type or metabolic profile.

17. The system, apparatus or method of any preceding or subsequent embodiment, wherein identifying a boundary comprises identifying a transition between pre-cancerous cells and benign cells.

18. The system, apparatus or method of any preceding or subsequent embodiment, wherein identifying a boundary comprises identifying a transition between cancerous cells and non-cancerous cells.

19. The system, apparatus or method of any preceding or subsequent embodiment: wherein the calibration image and decay image comprise an array of pixels across a field of view (FOV) of the target anatomy; and wherein the pixels in the array of pixels comprise fluorescence lifetime values that are acquired simultaneously across the FOV for both the calibration image and the decay image.

20. The system, apparatus or method of any preceding or subsequent embodiment, wherein said instructions, when executed by the processor, perform steps comprising: generating a reconstituted RGB image of the target anatomy; and displaying the reconstituted image simultaneously with the relative lifetime map of the target anatomy; wherein the a reconstituted RGB image and relative lifetime map are acquired using the same detector.

21. The system, apparatus or method of any preceding or subsequent embodiment, wherein the reconstituted RGB image is generated by acquiring separate images of the target anatomy by limiting acquisition of each image to only red, blue and green wavelengths within successive image captures on said detector, and then combining separate red, blue and green image captures to form the reconstituted RGB image.

22. The system, apparatus or method of any preceding or subsequent embodiment, wherein the relative lifetime map comprises a false color map of normalized fluorescence lifetime intensity across the array of pixels within the relative lifetime map.

23. The system, apparatus or method of any preceding or subsequent embodiment, wherein the relative lifetime map comprises pixel values that are proportional to an aggregate fluorophore decay time of the FOV.

24. The system, apparatus or method of any preceding or subsequent embodiment, wherein the FOV comprises a macroscopic FOV of the target anatomy.

25. The system, apparatus or method of any preceding or subsequent embodiment, wherein the excitation pulse comprises a pulse duration of approximately 30 ns.

26. The system, apparatus or method of any preceding or subsequent embodiment, wherein the array of LEDs comprises a circumferential array encircling the imaging lens so as to is illuminate target anatomy with the excitation pulse of light for a specified duration, wherein the array of LEDs focuses and multiplies illumination of the target anatomy across a FOV of the imaging lens.

27. The system, apparatus or method of any preceding or subsequent embodiment, wherein each of the LEDs in the array of LEDs comprises an aspherical lens to focus the excitation pulse of light across the FOV.

28. The system, apparatus or method of any preceding or subsequent embodiment, further comprising: (h) a diode driver coupled to the LED array; and (i) a pulse generator coupled to the diode driver and processor; (j) wherein the diode driver, pulse generator and LED array are coupled such that each of the array of LED's is configured to illuminate the FOV via non-sequential ray tracing.

29. A method for boundary detection within a target anatomy, the method comprising: (a) illuminating the target anatomy with an excitation pulse of light to excite fluorophores corresponding to the first tissue and second tissue; (b) acquiring a calibration image of the target anatomy during the excitation pulse, the calibration image comprising fluorescence lifetime values from emissions of the excited fluorophores; (d) acquiring a decay image of the target anatomy subsequent to the excitation pulse, the decay image comprising decayed fluorescence lifetime values as the emissions decay from bright to dark; (e) dividing the decay image by the calibration image to generate a relative lifetime map of the target anatomy; and (f) using the relative lifetime map, identifying a boundary between a first group of cells having a first physiologic process and a second group of cells having a second physiologic process; (g) wherein said method is performed by a processor executing instructions stored on a non-transitory medium.

30. The system, apparatus or method of any preceding or subsequent embodiment, wherein identifying a boundary comprises identifying a transition between cells of different aggregate type or metabolic profile.

31. The system, apparatus or method of any preceding or subsequent embodiment, wherein identifying a boundary comprises identifying a transition between pre-cancerous cells and benign cells.

32. The system, apparatus or method of any preceding or subsequent embodiment, wherein identifying a boundary comprises identifying a transition between cancerous cells and non-cancerous cells.

33. An apparatus for detecting cancerous cells within a target anatomy, comprising:(a) a processor; and (b) a non-transitory memory storing instructions executable by the processor; (c) wherein said instructions, when executed by the processor, perform steps comprising:(i) illuminating the target anatomy with a short pulse of light; (ii) measuring an intensity of a fluorescence emission from the target anatomy as the emission decays from bright to dark; and (iii) determining if a region within the target anatomy is cancerous or non-cancerous as a function of the fluorescence decay lifetime of the emission.

34. The system, apparatus or method of any preceding or subsequent embodiment, wherein said instructions when executed by the processor perform steps comprising: generating a false color map corresponding to measured decay lifetimes of the emissions.

35. A non-transitory medium storing instructions executable by a processor, said instructions when executed by the processor performing steps comprising: illuminating the target anatomy with a short pulse of light; measuring an intensity of a fluorescence emission from the target anatomy as the emission decays from bright to dark; and determining if a region within the target anatomy is cancerous or non-cancerous as a function of the fluorescence decay lifetime of the emission.

36. A method for detecting cancerous cells within a target anatomy, the method comprising: (a) illuminating the target anatomy with a short pulse of light; (b) measuring an intensity of a fluorescence emission from the target anatomy as the emission decays from bright to dark; and (c) determining if a region within the target anatomy is cancerous or non-cancerous as a function of the fluorescence decay lifetime of the emission; (d) wherein said method is performed by a processor executing instructions stored on a non-transitory medium.

37. The method of any preceding or following embodiment, wherein said instructions when executed by the processor perform steps comprising: generating a false color map corresponding to measured decay lifetimes of the emissions.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An apparatus for boundary detection within a target anatomy, comprising:
(a) a processor; and
(b) a non-transitory memory storing instructions executable by the processor;
(c) wherein said instructions, when executed by the processor, perform steps comprising:
illuminating the target anatomy with an excitation pulse of light to excite fluorophores corresponding to a first tissue and a second tissue;
(ii) acquiring a calibration image of the target anatomy during the excitation pulse, the calibration image comprising fluorescence values from emissions of the excited fluorophores;
(iii) acquiring a decay image of the target anatomy subsequent to the excitation pulse, the decay image comprising decayed fluorescence values as the emissions decay from bright to dark;
(iv) dividing the decay image by the calibration image to generate a relative lifetime map of the target anatomy; and
(v) using values in the relative lifetime map, identifying a boundary between a first group of cells having a first physiologic process and a second group of cells having a second physiologic process.

2. The apparatus of claim 1, wherein identifying a boundary comprises identifying a transition between cells of different aggregate type or metabolic profile.

3. The apparatus of claim 1, wherein identifying a boundary comprises identifying a transition between pre-cancerous cells and benign cells.

4. The apparatus of claim 1, wherein identifying a boundary comprises identifying a transition between cancerous cells and non-cancerous cells.

5. The apparatus of claim 1:
wherein the calibration image and decay image comprise an array of pixels across a field of view (FOV) of the target anatomy; and
wherein the pixels in the array of pixels comprise fluorescence lifetime values that are acquired simultaneously across the FOV for both the calibration image and the decay image.

6. The apparatus of claim 5, wherein said instructions, when executed by the processor, perform steps comprising:
generating a reconstituted RGB image of the target anatomy; and
displaying the reconstituted image simultaneously with the relative lifetime map of the target anatomy.

7. The apparatus of claim 5, wherein the reconstituted RGB image is generated by acquiring separate images of the target anatomy by limiting acquisition of each image to only red, blue and green wavelengths within successive image captures, and then combining separate red, blue and green image captures to form the reconstituted RGB image.

8. The apparatus of claim 5, wherein the relative lifetime map comprises a false color map of normalized fluorescence lifetime intensity across the array of pixels within the relative lifetime map.

9. The apparatus of claim 5, wherein the relative lifetime map comprises pixel values that are proportional to an aggregate fluorophore decay time of the FOV.

10. The apparatus of claim 5, wherein the FOV comprises a macroscopic FOV of the target anatomy.

11. The apparatus of claim 5, wherein the excitation pulse comprises a pulse duration of approximately 30 ns.

12. The apparatus of claim 1, further comprising:
(d) an imaging lens;
(e) an array of LEDs disposed at the front of the lens;
(f) wherein the array of LEDs is configured to illuminate target anatomy with the excitation pulse of light for a specified duration, wherein the array of LEDs focuses and multiplies illumination of the target anatomy across a FOV of the imaging lens; and
(g) a detector coupled to the imaging lens, the detector configured to acquire intensity data of the fluorescence emissions.

13. The apparatus of claim 12, wherein each of the LEDs in the array of LEDs comprises an aspherical lens to focus the excitation pulse of light across the FOV.

14. The apparatus of claim 12, further comprising:
(h) a diode driver coupled to the LED array; and
(i) a pulse generator coupled to the diode driver and processor;
(j) wherein the diode driver, pulse generator and LED array are coupled such that each of the array of LED's is configured to illuminate the FOV via non-sequential ray tracing.

15. A system for boundary detection within a target anatomy, the system comprising:
(a) an imaging lens;
(b) an array of LEDs disposed at or near the imaging lens;
(c) a detector coupled to the imaging lens, the detector configured to acquire intensity data of fluorescence emissions from the target anatomy;
(d) a processor coupled to the detector; and
(e) a non-transitory memory storing instructions executable by the processor;
(f) wherein said instructions, when executed by the processor, perform steps comprising:
(i) operating the array of LEDs to illuminate the target anatomy with an excitation pulse of light to excite fluorophores corresponding to a first tissue and a second tissue;
(ii) acquiring a calibration image of the target anatomy during the excitation pulse, the calibration image comprising fluorescence values from emissions of the excited fluorophores;
(iii) acquiring a decay image of the target anatomy subsequent to the excitation pulse, the decay image comprising decayed fluorescence values as the emissions decay from bright to dark;
(iv) dividing the decay image by the calibration image to generate a relative lifetime map of the target anatomy; and
(v) using values in the relative lifetime map, identifying a boundary between a first group of cells having a first physiologic process and a second group of cells having a second physiologic process.

16. The system of claim 15, wherein identifying a boundary comprises identifying a transition between cells of different aggregate type or metabolic profile.

17. The system of claim 15, wherein identifying a boundary comprises identifying a transition between pre-cancerous cells and benign cells.

18. The system of claim 15, wherein identifying a boundary comprises identifying a transition between cancerous cells and non-cancerous cells.

19. The system of claim 15:
wherein the calibration image and decay image comprise an array of pixels across a field of view (FOV) of the target anatomy; and
wherein the pixels in the array of pixels comprise fluorescence lifetime values that are acquired simultaneously across the FOV for both the calibration image and the decay image.

20. The apparatus of claim 19, wherein said instructions, when executed by the processor, perform steps comprising:
generating a reconstituted RGB image of the target anatomy; and
displaying the reconstituted image simultaneously with the relative lifetime map of the target anatomy;
wherein the a reconstituted RGB image and relative lifetime map are acquired using the same detector.

21. The apparatus of claim 20, wherein the reconstituted RGB image is generated by acquiring separate images of the target anatomy by limiting acquisition of each image to only red, blue and green wavelengths within successive image captures on said detector, and then combining separate red, blue and green image captures to form the reconstituted RGB image.

22. The system of claim 19, wherein the relative lifetime map comprises a false color map of normalized fluorescence lifetime intensity across the array of pixels within the relative lifetime map.

23. The system of claim 19, wherein the relative lifetime map comprises pixel values that are proportional to an aggregate fluorophore decay time of the FOV.

24. The system of claim 19, wherein the FOV comprises a macroscopic FOV of the target anatomy.

25. The system of claim 15, wherein the excitation pulse comprises a pulse duration of approximately 30 ns.

26. The system of claim 15, wherein the array of LEDs comprises a circumferential array encircling the imaging lens so as to is illuminate target anatomy with the excitation pulse of light for a specified duration, wherein the array of LEDs focuses and multiplies illumination of the target anatomy across a FOV of the imaging lens.

27. The system of claim 26, wherein each of the LEDs in the array of LEDs comprises an aspherical lens to focus the excitation pulse of light across the FOV.

28. The system of claim 26, further comprising:
(h) a diode driver coupled to the LED array; and
(i) a pulse generator coupled to the diode driver and processor;
(j) wherein the diode driver, pulse generator and LED array are coupled such that each of the array of LED's is configured to illuminate the FOV via non-sequential ray tracing.

29. A method for boundary detection within a target anatomy, the method comprising:
(a) illuminating the target anatomy with an excitation pulse of light to excite fluorophores corresponding to a first tissue and a second tissue;
(b) acquiring a calibration image of the target anatomy during the excitation pulse, the calibration image comprising fluorescence lifetime values from emissions of the excited fluorophores;
(d) acquiring a decay image of the target anatomy subsequent to the excitation pulse, the decay image comprising decayed fluorescence lifetime values as the emissions decay from bright to dark;
(e) dividing the decay image by the calibration image to generate a relative lifetime map of the target anatomy; and
(f) using the relative lifetime map, identifying a boundary between a first group of cells having a first physiologic process and a second group of cells having a second physiologic process;
(g) wherein said method is performed by a processor executing instructions stored on a non-transitory medium.

30. The method of claim 29, wherein identifying a boundary comprises identifying a transition between cells of different aggregate type or metabolic profile.

31. The method of claim 29, wherein identifying a boundary comprises identifying a transition between pre-cancerous cells and benign cells.

32. The method of claim 29, wherein identifying a boundary comprises identifying a transition between cancerous cells and non-cancerous cells.
